(19) United States
(12) Patent Application Publication
(10) Pub. No.: US 2023/0329984 A1
(43) Pub. Date: Oct. 19, 2023

(54) METHOD OF MAKING SHELF STABLE SULFUR CONTAINING PERSONAL CARE COMPOSITION

(71) Applicant: The Procter & Gamble Company, Cincinnati, OH (US)

(72) Inventors: James Robert-Paul Skallerup, Mason, OH (US); Brandon Scott Lane, Hamilton, OH (US); Marco Caggioni, Cincinnati, OH (US); Eric Scott Johnson, Hamilton, OH (US); Geoffrey Marc Wise, Reading, OH (US)

(21) Appl. No.: 18/298,458

(22) Filed: Apr. 11, 2023

Related U.S. Application Data
(60) Provisional application No. 63/330,934, filed on Apr. 14, 2022.

(51) Int. Cl. A61K 8/23; A61K 8/37; A61K 8/81; A61Q 5/02

(52) U.S. Cl. CPC A61K 8/23; A61K 8/375; A61K 8/8147; A61Q 5/02; A61K 2800/52

(57) ABSTRACT

The present invention is directed to a method for preparing a personal care composition comprising sulfur, wherein the method comprises a) Providing a composition comprising a surfactant, a stabilizing agent(s) and water, wherein the composition comprises at least about 3.0% surfactant and a stabilizing agent(s) sufficient to build a yield stress of at least 0.06 Pa; b) Mixing the composition in Step (a) wherein an agitation mechanism tip speed (as calculated by {agitator outer circumference [m/revolution]}*{agitator revolution rate [revolutions/s]}) is between about 1.5 m/s and about 2.7 m/s; with a recirculation turnover rate (as calculated by {recirculation pump rate [L/hour]}/{full turnover volume [L/turnover]}) of greater than 1.8 turnovers/hour; c) Adding sulfur to the composition of Step (b)—wherein the conditions of Step (b) are maintained while adding sulfur and until the personal care composition is prepared.

### FIELD OF THE INVENTION

The present invention is directed to making shelf stable personal care compositions comprising sulfur.

### BACKGROUND OF THE INVENTION

Conventionally, large hydrophobic particulates aggressively seek to separate from an aqueous personal care composition environment either by gravitational separation and/or by hydrophobic repulsion from the aqueous environment itself. In order to achieve shelf-stability, personal care compositions containing such particles will typically add them at very low concentrations (<0.1%) so as to minimize these effects and/or create tradeoffs by increasing product rheologies (>20,000 cPs at 2 s\hat{ }-1) to “cement” the particulate in place, which can lead to inhomogeneity of final formula without excessive mixing, such mixing itself can create instability. Such designs and making techniques can create undesirable making conditions e.g. too thick for dispensing or too expensive to process; and in the case of using Sulfur particulate (D50 ˜30 um) to treat dandruff, these tradeoffs are simply unacceptable and the shelf life of the product non-negotiable, thereby rendering these classic techniques unsuitable for its construction and in need of an improvement.

The present invention has found an order of operations prioritizing formula yield stress building, by means of the early inclusion of stability promoting agents, before sulfur addition and a discrete mix rate (as defined by tip speed) and a discrete recirculation rate (as defined by turnovers) afterword (balancing homogenization and stabilization) have-resulted-in making a shelf stable sulfur containing anti-dandruff shampoo. The degree with which this control is maintained is unexpected given the similarity of materials to other anti-dandruff shampoos using different anti-dandruff actives but having otherwise similar compositions which do not require these considerations. The benefit of each of these conditions can be demonstrated in the present invention.

### SUMMARY OF THE INVENTION

The present invention is directed to a method for preparing a personal care composition comprising sulfur, wherein the method comprises a) Providing a composition comprising a surfactant, a stabilizing agent(s) and water, wherein the composition comprises at least about 3.0% surfactant and a stabilizing agent(s) sufficient to build a yield stress of at least 0.06 Pa; b) Mixing the composition in Step (a) wherein an agitation mechanism tip speed (as calculated by {agitator outer circumference [m/revolution]}*{agitator revolution rate [revolutions/s]}) is between about 1.5 m/s and about 2.7 m/s; with a recirculation turnover rate (as calculated by {recirculation pump rate [L/hour]}/{full turnover volume [L/turnover]}) of greater than 1.8 turnovers/hour; c) Adding sulfur to the composition of Step (b)—wherein the conditions of Step (b) are maintained while adding sulfur and until the personal care composition is prepared.

### BRIEF DESCRIPTION OF THE DRAWINGS

FIG. 1 is a schematic diagram of a multi-tank process, including a premix tank and a tank, for preparing a personal care composition comprising sulfur.

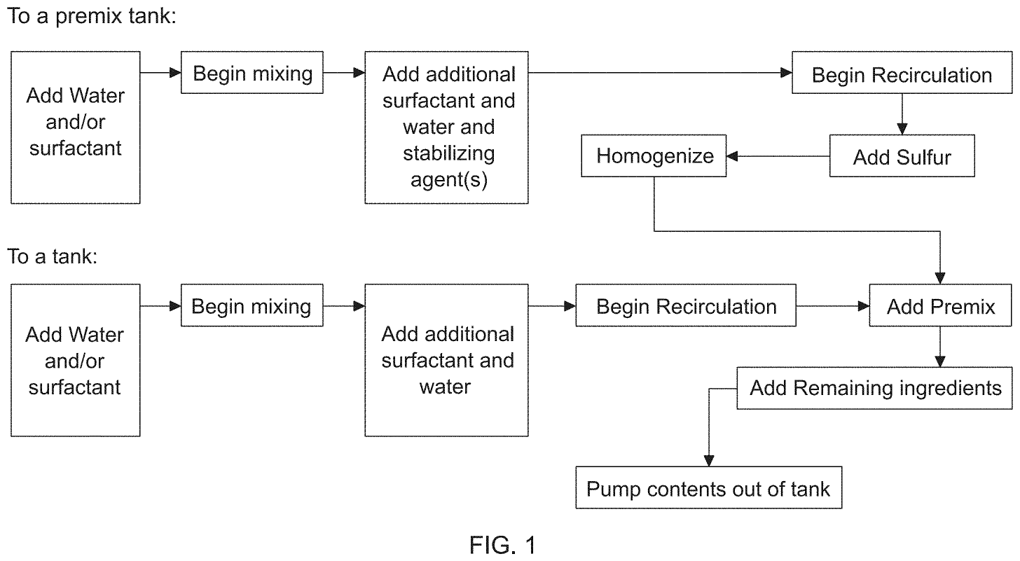

FIG. 2 is a schematic diagram of a single tank process for preparing a personal care composition comprising sulfur.

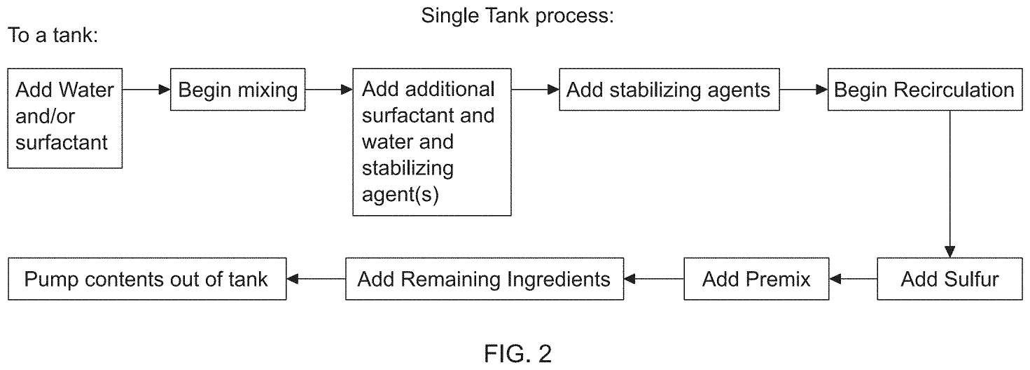

FIG. 3 is two photographs demonstrating examples of unincorporated sulfur left in a tank.

### DETAILED DESCRIPTION OF EMBODIMENTS OF THE INVENTION

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application,” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

While the specification concludes with claims particularly pointing out and distinctly claiming the invention, it is believed that the present invention will be better understood from the following description.

As used herein, the term “fluid” includes liquids and gels.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, “molecular weight” or “Molecular weight” refers to the weight average molecular weight unless otherwise stated. Molecular weight is measured using industry standard method, gel permeation chromatography (“GPC”).

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the hair care composition.

As used herein, “personal care compositions” includes products such as shampoos, shower gels, liquid hand cleansers, hair colorants, facial cleansers, and other surfactant-based liquid compositions As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Sulfur

The personal care composition of the present invention may include sulfur. The sulfur which is suitable for use herein can be any form of elemental sulfur. Sulfur exists at room temperatures primarily as rhombic crystals. The two most prevalent ways of obtaining elemental sulfur are: precipitation from hydrogen sulfide, with one route coming from contamination in sour gas, via the Claus process and mining underground deposits using superheated water, known as the Frasch process. Other forms of sulfur, such as monoclinic crystalline sulfur, oligomeric or polymeric sulfur, are the normal primary forms which elemental sulfur assumes at certain higher temperature ranges. At room temperatures, these forms convert, or revert, to rhombic sulfur. The sulfur while being in elemental form may be sulfur which has been physically mixed with protective colloids such as gum arabic, clays, waxes, oils, activated carbon, zeolites, silica or dispersing agents such as surfactants or subjected to processing steps to modify its particle size or other physical property. Sulfur is available commercially in a variety of forms such as pellets, cakes, prills, colloidal, micronized, sublimed, precipitated, and commercial flour.

Sulfur may have a particle size distribution wherein 90% of the particles (D90) of from about 30 micron (μm) to about 250 micron (μm); Sulfur may have a particle size distribution wherein the D90 is from about 30 micron (μm) to about 200 micron (μm); Sulfur may have a particle size distribution wherein the D90 is from about 30 micron (μm) to about 150 micron (μm); Sulfur may have a particle size distribution wherein the D90 is from about 30 micron (μm) to about 100 micron (μm).

Sulfur may have a particle size distribution wherein 50% of the particles (D50) is from about 5 micron (μm) to about 150 micron (μm); Sulfur may have a particle size distribution wherein the D50 is from about 10 micron (μm) to about 100 micron (μm); Sulfur may have a particle size distribution wherein the D50 is from about 15 micron (μm) to about 75 micron (μm); Sulfur may have a particle size distribution wherein the D50 is from about 20 micron (μm) to about 50 micron (μm).

Sulfur may have a particle size distribution wherein 10% of the particles (D10) is from about 1 micron (μm) to about 25 micron (μm); Sulfur may have a particle size distribution wherein the D10 is from 5 micron (μm) to about 25 micron (μm); Sulfur may have a particle size distribution wherein the D10 is from about 10 microns (μm) to about 25 micron (μm); Sulfur may have a particle size distribution wherein the D10 is from about 18 micron (μm) to about 25 micron (μm).

Sulfur may be present in a ratio of D(90)/D(10) of from about 3 to about 100; Sulfur may be present in a ratio of D(90)/D(10) of from about 3 to about 50; Sulfur may be present in a ratio of D(90)/D(10) of from about 3 to about 10; Sulfur may be present in a ratio of D(90)/D(10) of from about 3 to about 4.

The sulfur may be present in a final composition in an amount from about 0.1% to 5%, from about 0.25% to 4%, from about 0.5% to about 3%, and from about 0.5% to 2%. The sulfur may be present in the premix in an amount from about 2% to about 60%, from about 25% to about 60%, and from about 40% to about 55%,

Detersive Surfactant

The personal care composition may comprise greater than about 10% by weight of a surfactant system which provides cleaning performance to the composition and may be greater than 12% by weight of a surfactant system which provides cleaning performance to the composition. The surfactant system comprises an anionic surfactant and/or a combination of anionic surfactants and/or a combination of anionic surfactants and co-surfactants selected from the group consisting of amphoteric, zwitterionic, nonionic and mixtures thereof. Various examples and descriptions of detersive surfactants are set forth in U.S. Pat. No. 8,440,605; U.S. Patent Application Publication No. 2009/155383; and U.S. Patent Application Publication No. 2009/0221463, which are incorporated herein by reference in their entirety.

The personal care composition may comprise from about 10% to about 25%, from about 10% to about 18%, from about 10% to about 14%, from about 10% to about 12%, from about 11% to about 20%, from about 12% to about 20%, and/or from about 12% to about 18% by weight of one or more surfactants. The pre-mix composition may comprise from about 3% to about 20%, or from about 3% to about 9% by weight of one or more surfactants.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the hair care composition include ammonium lauryl sulfate, ammonium laureth sulfate, ammonium C10-15 pareth sulfate, ammonium C10-15 alkyl sulfate, ammonium C11-15 alkyl sulfate, ammonium decyl sulfate, ammonium deceth sulfate, ammonium undecyl sulfate, ammonium undeceth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, sodium C10-15 pareth sulfate, sodium C10-15 alkyl sulfate, sodium C11-15 alkyl sulfate, sodium decyl sulfate, sodium deceth sulfate, sodium undecyl sulfate, sodium undeceth sulfate, potassium lauryl sulfate, potassium laureth sulfate, potassium C10-15 pareth sulfate, potassium C10-15 alkyl sulfate, potassium C11-15 alkyl sulfate, potassium decyl sulfate, potassium deceth sulfate, potassium undecyl sulfate, potassium undeceth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. The anionic surfactant may be sodium lauryl sulfate or sodium laureth sulfate.

The composition of the present invention can also include anionic surfactants selected from the group consisting of:
a) R₁ O(CH₂CHR₃O)_{y} SO₃M;
b) CH₃ (CH₂)_{z} CHR₂ CH₂ 0 (CH₂ CHR₃O)_{y} SO₃M; and
c) mixtures thereof,
where R₁ represents CH₃ (CH₂)₁₀, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Suitable anionic alkyl sulfates and alkyl ether sulfate surfactants include, but are not limited to, those having branched alkyl chains which are synthesized from C8 to C18 branched alcohols which may be selected from the group consisting of: Guerbet alcohols, aldol condensation derived alcohols, oxo alcohols, F-T oxo alcohols and mixtures thereof. Non-limiting examples of the 2-alkyl branched alcohols include oxo alcohols such as 2-methyl-1-undecanol, 2-ethyl-1-decanol, 2-propyl-1-nonanol, 2-butyl 1-octanol, 2-methyl-1-dodecanol, 2-ethyl-1-undecanol, 2-propyl-1-decanol, 2-butyl-1-nonanol, 2-pentyl-1-octanol, 2-pentyl-1-heptanol, and those sold under the tradenames LIAL® (Sasol), ISALCHEM® (Sasol), and NEODOL® (Shell), and Guerbet and aldol condensation derived alcohols such as 2-ethyl-1-hexanol, 2-propyl-1-butanol, 2-butyl-1-octanol, 2-butyl-1-decanol, 2-pentyl-1-nonanol, 2-hexyl-1-octanol, 2-hexyl-1-decanol and those sold under the tradename ISOFOL® (Sasol) or sold as alcohol ethoxylates and alkoxylates under the tradenames LUTENSOL XP® (BASF) and LUTENSOL XL® (BASF).

The anionic alkyl sulfates and alkyl ether sulfates may also include those synthesized from C8 to C18 branched alcohols derived from butylene or propylene which are sold under the trade names EXXAL™ (Exxon) and Marlipal® (Sasol). This includes anionic surfactants of the subclass of sodium trideceth-n sulfates (STnS), where n is between about 0.5 and about 3.5. Exemplary surfactants of this subclass are sodium trideceth-2 sulfate and sodium trideceth-3 sulfate. The composition of the present invention can also include sodium tridecyl sulfate.

The composition of the present invention can also include anionic alkyl and alkyl ether sulfosuccinates and/or dialkyl and dialkyl ether sulfosuccinates and mixtures thereof. The dialkyl and dialkyl ether sulfosuccinates may be a C6-15 linear or branched dialkyl or dialkyl ether sulfosuccinate. The alkyl moieties may be symmetrical (i.e., the same alkyl moieties) or asymmetrical (i.e., different alkyl moieties). Nonlimiting examples include: disodium lauryl sulfosuccinate, disodium laureth sulfosuccinate, sodium bistridecyl sulfosuccinate, sodium dioctyl sulfosuccinate, sodium dihexyl sulfosuccinate, sodium dicyclohexyl sulfosuccinate, sodium diamyl sulfosuccinate, sodium diisobutyl sulfosuccinate, linear bis(tridecyl) sulfosuccinate and mixtures thereof.

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

“Substantially free” of sulfate based surfactants as used herein means from about 0 wt % to about 3 wt %, alternatively from about 0 wt % to about 2 wt %, alternatively from about 0 wt % to about 1 wt %, alternatively from about 0 wt % to about 0.5 wt %, alternatively from about 0 wt % to about 0.25 wt %, alternatively from about 0 wt % to about 0.1 wt %, alternatively from about 0 wt % to about 0.05 wt %, alternatively from about 0 wt % to about 0.01 wt %, alternatively from about 0 wt % to about 0.001 wt %, and/or alternatively free of sulfates. As used herein, “free of” means 0 wt %.

The hair care composition may comprise a co-surfactant. The co-surfactant can be selected from the group consisting of amphoteric surfactant, zwitterionic surfactant, non-ionic surfactant and mixtures thereof. The co-surfactant can include, but is not limited to, lauramidopropyl betaine, cocoamidopropyl betaine, lauryl hydroxysultaine, sodium lauroamphoacetate, disodium cocoamphodiacetate, cocamide monoethanolamide and mixtures thereof.

The hair care composition may further comprise from about 0.25% to about 15%, from about 1% to about 14%, from about 2% to about 13% by weight of one or more amphoteric, zwitterionic, nonionic co-surfactants, or a mixture thereof.

Suitable amphoteric or zwitterionic surfactants for use in the hair care composition herein include those which are known for use in shampoo or other hair care cleansing. Non limiting examples of suitable zwitterionic or amphoteric surfactants are described in U.S. Pat. Nos. 5,104,646 and 5,106,609, which are incorporated herein by reference in their entirety.

Amphoteric co-surfactants suitable for use in the composition include those surfactants described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable amphoteric surfactant include, but are not limited to, those selected from the group consisting of: sodium cocaminopropionate, sodium cocaminodipropionate, sodium cocoamphoacetate, sodium cocoamphodiacetate, sodium cocoamphohydroxypropylsulfonate, sodium cocoamphopropionate, sodium cornamphopropionate, sodium lauraminopropionate, sodium lauroamphoacetate, sodium lauroamphodiacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphodiacetate, ammonium cocoamphohydroxypropylsulfonate, ammonium cocoamphopropionate, ammonium cornamphopropionate, ammonium lauraminopropionate, ammonium lauroamphoacetate, ammonium lauroamphodiacetate, ammonium lauroamphohydroxypropylsulfonate, ammonium lauroamphopropionate, ammonium cornamphopropionate, ammonium lauriminodipropionate, triethanolamine cocaminopropionate, triethanolamine cocaminodipropionate, triethanolamine cocoamphoacetate, triethanolamine cocoamphohydroxypropylsulfonate, triethanolamine cocoamphopropionate, triethanolamine cornamphopropionate, triethanolamine lauraminopropionate, triethanolamine lauroamphoacetate, triethanolamine lauroamphohydroxypropylsulfonate, triethanolamine lauroamphopropionate, triethanolamine cornamphopropionate, triethanolamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof.

The composition may comprises a zwitterionic co-surfactant, wherein the zwitterionic surfactant is a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof.

Suitable nonionic surfactants for use in the present invention include those described in McCutcheion's Detergents and Emulsifiers, North American edition (1986), Allured Publishing Corp., and McCutcheion's Functional Materials, North American edition (1992). Suitable nonionic surfactants for use in the personal care compositions of the present invention include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

The co-surfactant can be a non-ionic surfactant selected from the alkanolamides group including: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, PPG-2 Hydroxyethyl Isostearamide and mixtures thereof.

Representative polyoxyethylenated alcohols include alkyl chains ranging in the C9-C16 range and having from about 1 to about 110 alkoxy groups including, but not limited to, laureth-3, laureth-23, ceteth-10, steareth-10, steareth-100, beheneth-10, and commercially available from Shell Chemicals, Houston, Texas under the trade names Neodol® 91, Neodol® 23, Neodol® 25, Neodol® 45, Neodol® 135, Neodol® 67, Neodol® PC 100, Neodol® PC 200, Neodol® PC 600, and mixtures thereof.

Also available commercially are the polyoxyethylene fatty ethers available commercially under the Brij® trade name from Uniqema, Wilmington, Delaware, including, but not limited to, Brij® 30, Brij® 35, Brij® 52, Brij® 56, Brij® 58, Brij® 72, Brij® 76, Brij® 78, Brij® 93, Brij® 97, Brij® 98, Brij® 721 and mixtures thereof.

Suitable alkyl glycosides and alkyl polyglucosides can be represented by the formula (S)n-O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, lauryl alcohol, myristyl alcohol, cetyl alcohol, stearyl alcohol, oleyl alcohol, and the like. Examples of these surfactants include alkyl polyglucosides wherein S is a glucose moiety, R is a C8-20 alkyl group, and n is an integer of from about 1 to about 9. Commercially available examples of these surfactants include decyl polyglucoside and lauryl polyglucoside available under trade names APG® 325 CS, APG® 600 CS and APG® 625 CS) from Cognis, Ambler, Pa. Also useful herein are sucrose ester surfactants such as sucrose cocoate and sucrose laurate and alkyl polyglucosides available under trade names Triton™ BG-10 and Triton™ CG-110 from The Dow Chemical Company, Houston, Tx.

Other nonionic surfactants suitable for use in the present invention are glyceryl esters and polyglyceryl esters, including but not limited to, glyceryl monoesters, glyceryl monoesters of C12-22 saturated, unsaturated and branched chain fatty acids such as glyceryl oleate, glyceryl monostearate, glyceryl monopalmitate, glyceryl monobehenate, and mixtures thereof, and polyglyceryl esters of C12-22 saturated, unsaturated and branched chain fatty acids, such as polyglyceryl-4 isostearate, polyglyceryl-3 oleate, polyglyceryl-2-sesquioleate, triglyceryl diisostearate, diglyceryl monooleate, tetraglyceryl monooleate, and mixtures thereof.

Also useful herein as nonionic surfactants are sorbitan esters. Sorbitan esters of C12-22 saturated, unsaturated, and branched chain fatty acids are useful herein. These sorbitan esters usually comprise mixtures of mono-, di-, tri-, etc. esters. Representative examples of suitable sorbitan esters include sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), and sorbitan isostearate.

Also suitable for use herein are alkoxylated derivatives of sorbitan esters including, but not limited to, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), and mixtures thereof, all available from Uniqema.

Also suitable for use herein are alkylphenol ethoxylates including, but not limited to, nonylphenol ethoxylates (Tergitol™ NP-4, NP-6, NP-7, NP-8, NP-9, NP-10, NP-11, NP-12, NP-13, NP-15, NP-30, NP-40, NP-50, NP-55, NP-70 available from The Dow Chemical Company, Houston, Tx.) and octylphenol ethoxylates (Triton™ X-15, X-35, X-45, X-114, X-100, X-102, X-165, X-305, X-405, X-705 available from The Dow Chemical Company, Houston, TX).

Also suitable for use herein are tertiary alkylamine oxides including lauramine oxide and cocamine oxide.

Non limiting examples of other anionic, zwitterionic, amphoteric, and non-ionic additional surfactants suitable for use in the hair care composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

Suitable surfactant combinations comprise an average weight % of alkyl branching of from about 0.5% to about 30%, alternatively from about 1% to about 25%, alternatively from about 2% to about 20%. The surfactant combination can have a cumulative average weight % of C8 to C12 alkyl chain lengths of from about 7.5% to about 25%, alternatively from about 10% to about 22.5%, alternatively from about 10% to about 20%. The surfactant combination can have an average C8-C12/C13-C18 alkyl chain ratio from about 3 to about 200, alternatively from about 25 to about 175.5, alternatively from about 50 to about 150, alternatively from about 75 to about 125.

Stabilizing Polymers

The personal care composition can comprise a stabilizing polymer to increase the viscosity or yield stress of the composition. Suitable stabilizing polymers can be used. The hair care composition can comprise from about 0.05% to 10% and 0.1% to about 9% of a stabilizing, from about 0.4% to about 8% of a stabilizing polymer, from about 0.7% to about 5% of a stabilizing modifying polymer, and from about 1% to about 2.5% of a stabilizing polymer. The stabilizing polymer modifier may be a polyacrylate, polyacrylamide thickeners. The stabilizing polymer may be an anionic stabilizing polymer. A sulfur containing premix will contain a composition of about 0.1% to about 9%.

The personal care composition may comprise stabilizing polymers that are homopolymers based on acrylic acid, methacrylic acid or other related derivatives, non-limiting examples include polyacrylate, polymethacrylate, polyethylacrylate, and polyacrylamide.

The stabilizing polymers may be alkali swellable and hydrophobically-modified alkali swellable acrylic copolymers or methacrylate copolymers, non-limiting examples include acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer.

The stabilizing polymer may be soluble crosslinked acrylic polymers, a non-limiting example includes carbomers.

The stabilizing polymer may be an associative polymeric thickeners, non-limiting examples include: hydrophobically modified, alkali swellable emulsions, non-limiting examples include hydrophobically modified polypolyacrylates; hydrophobically modified polyacrylic acids, and hydrophobically modified polyacrylamides; hydrophobically modified polyethers wherein these materials may have a hydrophobe that can be selected from cetyl, stearyl, oleayl, and combinations thereof.

The stabilizing polymer may be used in combination with polyvinylpyrrolidone, crosslinked polyvinylpyrrolidone and derivatives. The stabilizing polymer may be combined with polyvinyalcohol and derivatives. The stabilizing polymer may be combined with polyethyleneimine and derivatives.

The stabilizing polymers may be combined with alginic acid based materials, non-limiting examples include sodium alginate, and alginic acid propylene glycol esters.

The stabilizing polymer may be used in combination with polyurethane polymers, non-limiting examples include: hydrophobically modified alkoxylated urethane polymers, non-limiting examples include PEG-150/decyl alcohol/SMDI copolymer, PEG-150/stearyl alcohol/SMDI copolymer, polyurethane-39.

The stabilizing polymer may be combined with an associative polymeric thickeners, non-limiting examples include: hydrophobically modified cellulose derivatives; and a hydrophilic portion of repeating ethylene oxide groups with repeat units from 10-300, from 30-200, and from 40-150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

The stabilizing polymer may be combined with cellulose and derivatives, including cellulose gums, non-limiting examples include microcrystalline cellulose, carboxymethylcelluloses, hydroxyethylcellulose, hydroxypropylcellulose, hydroxypropyl methylcellulose, methylcellulose, ethyl cellulose; nitro cellulose; cellulose sulfate; cellulose powder; hydrophobically modified celluloses.

The stabilizing polymer may be combined with a guar and guar derivatives, non-limiting examples include hydroxypropyl guar, and hydroxypropyl guar hydroxypropyl trimonium chloride.

The stabilizing polymer may be combined with polyethylene oxide; polypropylene oxide; and POE-PPO copolymers.

The stabilizing polymer may be combined with polyalkylene glycols characterized by the general formula:

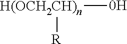

wherein R is hydrogen, methyl, or mixtures thereof, preferably hydrogen, and n is an integer having an average from 2,000-180,000, or from 7,000-90,000, or from 7,000-45,000. Non-limiting examples of this class include PEG-7M, PEG-14M, PEG-23M, PEG-25M, PEG-45M, PEG-90M, or PEG-100M.

The stabilizing polymer may be combined with silicas, non-limiting examples include fumed silica, precipitated silica, and silicone-surface treated silica.

The stabilizing polymer may be combined with water-swellable clays, non-limiting examples include laponite, bentolite, montmorilonite, smectite, and hectonite.

The stabilizing polymer may be combined with gums, non-limiting examples include xanthan gum, guar gum, hydroxypropyl guar gum, Arabia gum, tragacanth, galactan, carob gum, karaya gum, and locust bean gum.

The stabilizing polymer may be combined with, dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran,

Non-limiting examples of stabilizing polymer include acrylamide/ammonium acrylate copolymer (and) polyisobutene (and) polysorbate 20; acrylamide/sodium acryloyldimethyl taurate copolymer/isohexadecane/polysorbate 80, ammonium acryloyldimethyltaurate/VP copolymer, Sodium Acrylate/Sodium Acryloyldimethyl Taurate Copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; carbomer, sodium carbomer, crosslinked polyvinylpyrrolidone (PVP), polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and) trideceth-6, Acrylamide/Sodium Acryloyldimethyltaurate/Acrylic Acid Copolymer, sodium acrylate/acryloyldimethyltaurate/dimethylacrylamide, crosspolymer (and) isohexadecane (and) polysorbate 60, sodium polyacrylate. Exemplary commercially-available stabilizing polymers include ACULYN™ 28, ACULYN™ 88, ACULYN™ 33, ACULYN™ 22, ACULYN™ Excel, Carbopol® Aqua SF-1, Carbopol® ETD 2020, Carbopol® Ultrez 20, Carbopol® Ultrez 21, Carbopol® Ultrez 10, Carbopol® Ultrez 30, Carbopol® 1342, Carbopol® Aqua SF-2 Polymer, Sepigel™ 305, Simulgel™ 600, Sepimax Zen, Carbopol® SMART 1000, Rheocare® TTA, Rheomer® SC-Plus, STRUCTURE@ PLUS, Aristoflex® AVC, Stabylen 30, and combinations thereof.

### Suspending Wax

Suspending waxes includes suitable stabilizing agents that increase yield stress and viscosity. Such materials may include monoester and/or diester of alkylene glycols having the formula:

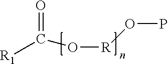

wherein R₁ is linear or branched C12-C22 alkyl group;
R is linear or branched C2-C4 alkylene group;
P is selected from H, C1-C4 alkyl or —COR₂, R₂ is C4-C22 alkyl, or may be C12-C22 alkyl; and n=1-3.

In the present invention, the long chain fatty ester may have the general structure described above, wherein R₁ is linear or branched C16-C22 alkyl group, R is —CH₂—CH₂—, and P is selected from H, or —COR₂, wherein R₂ is C4-C22 alkyl, or may be C12-C22 alkyl.

Typical examples are monoesters and/or diesters of ethylene glycol, propylene glycol, diethylene glycol, dipropylene glycol, triethylene glycol or tetraethylene glycol with fatty acids containing from about 6 to about 22, from about 12 to about 18 carbon atoms, such as caproic acid, caprylic acid, 2-ethyhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, arachic acid, gadoleic acid, behenic acid, erucic acid, and mixtures thereof.

In the present invention, ethylene glycol monostearate (EGMS) and/or ethylene glycol distearate (EGDS) and/or polyethylene glycol monostearate (PGMS) and/or polyethyleneglycol distearate (PGDS) may be suspending waxes used in the composition. There are several commercial sources for these materials. For Example, PEG6000MS® is available from Stepan, Empilan EGDS/A® is available from Albright & Wilson.

Traditionally glyceride ester compounds may be used as a structurant for personal care compositions. For example, Thixcin® R is trihydroxystearin, a commercial hydrogenated castor oil produced by Elementis Specialties of New Jersey, and marketed as a stabilizer and structurant for personal care compositions. Suitable glyceride esters for the personal care compositions described herein can be selected from any crystallizable glyceride esters which can allow for the formation of a coacervate in personal care compositions including a suitable surfactant and a cationic polymer. For example, suitable glyceride esters are hydrogenated castor oils such as trihydroxystearin or dihydroxystearin.

Examples of additional crystallizable glyceride esters can include the substantially pure triglyceride of 12-hydroxystearic acid. 12-hydroxystearic acid is the pure form of a fully hydrogenated triglyceride of 12-hydrox-9-cis-octadecenoic acid. As can be appreciated, many additional glyceride esters are possible. For example, variations in the hydrogenation process and natural variations in castor oil can enable the production of additional suitable glyceride esters from castor oil.

Suitable glyceride esters can also be formed from mixtures of one or more glycerides. For example, a mixture of glycerides including about 80% or more, by weight of the mixture, castor oil, can be suitable. Other suitable mixtures can include mixtures of only triglycerides, mixtures of diglycerides and triglycerides, mixtures of triglycerides with diglycerides and limited amounts, e.g., less than about 20%, by weight of the mixture, of monoglyerides; or any mixture thereof which includes about 20% or less, by weight of the mixture, of a corresponding acid hydrolysis product of any of the glycerides. About 80% or more, by weight of a mixture, can be chemically identical to a glyceride of fully hydrogenated ricinoleic acid, i.e., glyceride of 12-hydroxystearic acid. Hydrogenated castor oil can be modified such that in a given triglyceride, there will be two 12-hydroxystearic moieties and one stearic moiety. Alternatively, partial hydrogenation can be used. However, poly(oxyalkylated) castor oils are not suitable because they have unsuitable melting points.

Castor oils include glycerides, especially triglycerides, comprising C10 to C 22 alkyl or alkenyl moieties which incorporate a hydroxyl group. Hydrogenation of castor oil produces hydrogenated castor oil by converting double bonds, which are present in the starting oil as ricinoleyl moieties. These moieties are converted to ricinoleyl moieties, which are saturated hydroxyalkyl moieties, e.g., hydroxystearyl. The hydrogenated castor oil (HCO) herein may, be selected from: trihydroxystearin; dihydroxystearin; and mixtures thereof. The HCO may be processed in any suitable starting form, including, but not limited those selected from solid, molten and mixtures thereof. Useful HCO may have the following characteristics: a melting point of from about 40° C. to about 100° C., alternatively from about 65° C. to about 95° C.; and/or Iodine value ranges of from about 0 to about 5, alternatively from about 0 to about 4, and alternatively from about 0 to about 2.6. The melting point of HCO can measured using DSC: Differential Scanning Calorimetry.

Suitable HCO include those that are commercially available. Non-limiting examples of commercially available HCO suitable for use include: THIXCIN-R® (supplied by Elementis), which is supplied as a powder having small particles (99 weight % smaller than of 44 micrometers).

The invention is not intended to be directed only to the use of hydrogenated castor oil. Any other suitable crystallizable glyceride may be used. In one example, the structurant is substantially pure triglyceride of 12-hydroxystearic acid. This molecule represents the pure form of a fully hydrogenated triglyceride of 12-hydrox-9-cis-octadecenoic acid. In nature, the composition of castor oil may vary somewhat. Likewise hydrogenation procedures may vary. Any other suitable equivalent materials, such as mixtures of triglycerides wherein at least about 80% wt. is from castor oil, may be used. Exemplary equivalent materials comprise primarily, or consist of, triglycerides; or comprise primarily, or consist of, mixtures of diglycerides and triglycerides; or comprise primarily, or consist of, mixtures of triglyerides with diglycerides and limited amounts, e.g., less than about 20% wt. of the glyceride mixtures, of monoglyerides; or comprise primarily, or consist of, any of the foregoing glycerides with limited amounts, e.g., less than about 20% wt., of the corresponding acid hydrolysis product of any of said glycerides.

The stabilizing premix comprises from about 4% to about 30% by weight of the personal care composition of a 100% active stabilizing agent. In the present invention, the stabilizing premix may comprise from about 15% to about 25% of stabilizing agent.

The suspending wax may be in the present invention from about 0.01% to about 4%; the suspending wax may be in the present invention from about 0.1% to about 3%; the suspending wax may be in the present invention from about 0.5% to about 2%; suspending wax may be in the present invention from about 0.3% to about 1.5%.

Water Miscible Solvents

The carrier of the personal care composition may include water and water solutions of lower alkyl alcohols, polyhydric alcohols, ketones having from 3 to 4 carbons atoms, C1-C6 esters of C1-C6 alcohols, sulfoxides, amides, carbonate esters, ethoxylated and proposylated C1-C10 alcohols, lactones, pyrollidones, and mixtures thereof. Non-limited lower alkyl alcohol examples are monohydric alcohols having 1 to 6 carbons, such as ethanol and isopropanol. Non-limiting examples of polyhydric alcohols useful herein include propylene glycol, dipropylene glycol, butylenes glycol, hexylene glycol, glycerin, propane diol and mixtures thereof.

In present invention, the hair care composition may comprise a hydrotrope/viscosity modifier which is an alkali metal or ammonium salt of a lower alkyl benzene sulphonate such as sodium xylene sulphonate, sodium cumene sulphonate or sodium toluene sulphonate.

In the present invention, the hair care composition may comprise silicone/PEG-8 silicone/PEG-9 silicone/PEG-n silicone/silicone ether (n could be another integer), non-limiting examples include PEG8-dimethicone A208) MW 855, PEG 8 Dimethicone D208 MW 2706.

Optional Ingredients

In the present invention, the personal care composition may further comprise one or more optional ingredients, including scalp health agents and benefit agents. Suitable scalp health agents and benefit agents include, but are not limited to conditioning agents, cationic polymers, silicone emulsions, anti-dandruff agents such as polyvalent metal salts of pyrithione, non-limiting examples include zinc pyrithione (ZPT) and copper pyrithione, or selenium sulfide, benefit agents providing moisturization, barrier improvement, anti-fungal, anti-microbial and anti-oxidant, anti-itch, and sensates, gel networks, chelating agents, and natural oils such as sun flower oil or castor oil. Additional suitable optional ingredients include but are not limited to perfumes, perfume microcapsules, colorants, particles, anti-microbials, foam busters, anti-static agents, rheology modifiers and thickeners, emulsifiers, suspension materials and structurants, pH adjusting agents and buffers, preservatives, pearlescent agents, solvents, diluents, anti-oxidants, vitamins, vitamin E and F, salicylic acid, niacinamide, caffeine, panthenol, zinc oxide, zinc carbonate, basic zinc carbonate, glycols, glycolic acid, PCA, PEGs, erythritol, glycerin, triclosan, lactates, hyaluronates, allantoin and other ureas, betaines, sorbitol, glutamates, xylitols, menthol, menthyl lactate, iso cyclomone, benzyl alcohol, coal tar, charcoal, whitfield's ointment, castellani's paint, aluminum chloride, gentian violet, octopirox (piroctone olamine), ciclopirox olamine, undecylenic acid and its metal salts, azoxystrobin and other strobulins, potassium permanganate, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, itraconazole, ketoconazole benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, terconazole and combinations thereof. In the present invention, a perfume may be present from about 0.5% to about 7%.

One or more stabilizers can be included. For example, one or more of ethylene glycol distearate, citric, citrate, a preservative such as kathon, sodium chloride, sodium benzoate, and ethylenediaminetetraacetic acid (“EDTA”) can be included to improve the lifespan of a personal care composition.

Such optional ingredients should be physically and chemically compatible with the components of the composition, and should not otherwise unduly impair product stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to the composition herein.

Chelating Agents

The personal care composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. No. 5,747,440.

Chelating agents can be incorporated in the compositions herein in amounts ranging from 0.001% to 10.0% by weight of the total composition, preferably 0.01% to 2.0%.

Nonlimiting chelating agent classes include carboxylic acids, aminocarboxylic acids, including aminocids, phosphoric acids, phosphonic acids, polyphosponic acids, polyethyleneimines, polyfunctionally-substituted aromatic, their derivatives and salts.

Nonlimiting chelating agents include the following materials and their salts. Ethylenediaminetetraacetic acid (EDTA), ethylenediaminetriacetic acid, ethylenediamine-N,N′-disuccinic acid (EDDS), ethylenediamine-N,N′-diglutaric acid (EDDG), salicylic acid, aspartic acid, glutamic acid, glycine, malonic acid, histidine, diethylenetriaminepentaacetate (DTPA), N-hydroxyethylethylenediaminetriacetate, nitrilotriacetate, ethylenediaminetetrapropionate, triethylenetetraaminehexaacetate, ethanoldiglycine, propylenediaminetetracetic acid (PDTA), methylglycinediacetic acid (MODA), diethylenetriaminepentaacetic acid, methylglycinediacetic acid (MGDA), N-acyl-N,N′,N′-ethylenediaminetriacetic acid, nitrilotriacetic acid, ethylenediaminediglutaric acid (EDGA), 2-hydroxypropylenediamine disuccinic acid (HPDS), glycinamide-N, N′-disuccinic acid (GADS), 2-hydroxypropylenediamine-N—N′-disuccinic acid (HPDDS), N-2-hydroxyethyl-N,N-diacetic acid, glyceryliminodiacetic acid, iminodiacetic acid-N-2-hydroxypropyl sulfonic acid, aspartic acid N-carboxymethyl-N-2-hydroxypropyl-3-sulfonic acid, alanine-N,N′-diacetic acid, aspartic acid-N,N′-diacetic acid, aspartic acid N-monoacetic acid, iminodisuccinic acid, diamine-N,N′-dipolyacid, monoamide-N,N′-dipolyacid, diaminoalkyldi(sulfosuccinic acids) (DDS), ethylenediamine-N—N′-bis (ortho-hydroxyphenyl acetic acid)), N,N′-bis(2-hydroxybenzyl)ethylenediamine-N, N′-diacetic acid, ethylenediaminetetraproprionate, triethylenetetraaminehexacetate, diethylenetriaminepentaacetate, dipicolinic acid, ethylenedicysteic acid (EDC), ethylenediamine-N,N′-bis(2-hydroxyphenylacetic acid) (EDDHA), glutamic acid diacetic acid (GLDA), hexadentateaminocarboxylate (HBED), polyethyleneimine, 1-hydroxydiphosphonate, aminotri(methylenephosphonic acid) (ATMP), nitrilotrimethylenephosphonate (NTP), ethylenediaminetetramethylenephosphonate, diethylenetriaminepentamethylenephosphonate (DTPMP), ethane-1-hydroxydiphosphonate (HEDP), 2-phosphonobutane-1,2,4-tricarboxylic acid, polvphosphoric acid, sodium tripolyphosphate, tetrasodium diphosphate, hexametaphosphoric acid, sodium metaphosphate, phosphonic acid and derivatives, Aminoalkylen-poly(alkylenphosphonic acid), aminotri(1-ethylphosphonic acid), ethylenediaminetetra(1-ethylphosphonic acid), aminotri(1-propylphosphonic acid), aminotri(isopropylphosphonic acid), ethylenediaminetetra(methylenephosphonic acid) (EDTMP), 1,2-dihydroxy-3,5-disulfobenzene.

### Aqueous Carrier

The personal care compositions can be in the form of pourable liquids (under ambient conditions). Such compositions will therefore typically comprise a carrier, which is present at a level of from about 40% to about 85%, alternatively from about 45% to about 80%, alternatively from about 50% to about 75% by weight of the hair care composition. The carrier may comprise water, or a miscible mixture of water and organic solvent, and in one aspect may comprise water with minimal or no significant concentrations of organic solvent, except as otherwise incidentally incorporated into the composition as minor ingredients of other essential or optional components.

The carrier useful in the personal care compositions of the present invention may include water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

Product Form

The personal care compositions of the present invention may be presented in typical personal care formulations. They may be in the form of solutions, dispersion, emulsions, powders, talcs, encapsulated, spheres, spongers, solid dosage forms, foams, and other delivery mechanisms. The compositions of the present invention may be hair tonics, leave-on hair products such as treatment, and styling products, rinse-off hair products such as shampoos and personal cleansing products, and treatment products; and any other form that may be applied to hair.

Methods

### Viscosity Measurement

Personal care composition viscosities can be measured on a 2.5 mL sample using a cone and plate Brookfield RS rheometer with cone C75-1 at constant shear rate of 2 s⁻¹, at 27° C. at 3 mins. In the present invention the viscosity of the prepared personal care composition may be <20,000 cPs @ 2 s\hat{ }-1. The viscosity of the prepared personal care composition may be in the range of 8000-14000 cPs @ 2 s\hat{ }-1.

Measurement of Sulfur Particle Size Distribution Sulfur particle size distribution is measured with a Horiba LA-950 Laser Scattering Particle Size Distribution Analyzer with LA-950 for Windows software version 8.10. Sulfur material is continuously stirred in 1% SDS solution until uniformly dispersed. While continuing to stir, the dispersed sulfur is transferred to the Horiba instrument into a dispersant of 0.1% SDS with circulation (speed: 3), agitation (speed: 1), and sonication (power: 1) turned on. The dispersed sulfur sample is added until percent transmittance is about 90%. Refractive index values of 2.245-0.10i for sulfur and 1.333 for the dispersant are used to report D10, D50, and D90 values on a volume basis.

Non-limiting examples of a conventional agitation mechanism would be a pitch blade turbine, helicoidal mixer, and a tank sweeping mixing or an edge sweeping mixer.

In the present invention, a method for preparing a personal care composition comprising sulfur, may comprise the following steps:
a) Providing a composition comprising a surfactant, a stabilizing agent(s) and water, wherein the composition comprises at least about 3.0% surfactant and a stabilizing agent(s) sufficient to build a yield stress of at least 0.06 Pa;
b) Mixing the composition in Step (a) wherein an agitation mechanism's tip speed (as calculated by {agitator outer circumference [m/revolution]}*{agitator revolution rate [revolutions/s]}) is between about 1.5 m/s and about 2.7 m/s; with a recirculation turnover rate (as calculated by {recirculation pump rate [L/hour]}/{full turnover volume [L/turnover]}) of greater than 1.8 turnovers/hour;
c) Adding sulfur to the composition of Step (b)—wherein the conditions of Step b are maintained while adding sulfur and until the personal care composition is prepared.
   In the present invention the premixture may have a temperature of not greater than about 60° C.

An agitation mechanism tip speed may be conventionally calculated by the equation: {agitator outer circumference [m/revolution]}*{agitator revolution rate [revolutions/s]. The agitator outer circumference may be a function of the radius or diameter. An agitator revolution rate may be the way mechanical speeds are given. Revolutions per minute or RPM may be an industry standard method for measuring rates for equipment or a device. Conventionally, a diameter may be marked on the device or provided in an equipment or device specification. The revolution rate will be part of the operational condition of the equipment or device and the method by which its operation is governed. A non-limiting example may be a mixer which has a knob that can be used to set at a speed, that set speed being an RPM wherein the RPM is what is being manipulated to actually work the equipment.

A recirculation turnover rate may be conventionally calculated by the equation: {recirculation pump rate [L/hour]}/{full turnover volume [L/turnover]}. The pump rate may be measured by use of equipment or device to measure the rate. A nonlimiting example would be using a flowmeter in a pump line path. The turnover volume may be the full volume of the personal care composition. A nonlimiting example would be for the making of a 50 L batch, then the full volume would be 50 L.

### Yield Stress Table and Yield Stress Determination Method

To perform these measurements, the present invention uses a TA DHR3 rheometer equipped with a cone and plate geometry.

A solvent trap is added to avoid sample evaporation during the measurement. This uses a cone and plate geometry, run up to 40° C. and lasts about 30 min.

Test 1—Flow Curve at 25° C.: steady state flow curve in strain controlled mode starting at 100 1/s, down to 0.01 1/s, performed at a fixed temperature of 25° C. The choice to control the shear rate and to start at high shear is made in order to measure the dynamic yield stress. This measurement protocol minimizes the effect of the sample thixotropy which could induce variability in the measurement of the yield stress based on sample shear history. The total duration of this test ˜8 min.

Test 2—Temperature ramp: temperature ramp at fixed shear rate of 2 s-1, starting from 25° C. up to 40° C. The choice of 2 s-1 shear rate is made because it captures the value of the low-shear plateau of the worm like micellar microstructure present in the clear phase. The total duration of this test ˜8 min.

Test 3—Flow Curve at 40° C.: steady state flow curve in strain controlled mode starting at 100 1/s down to 0.01 1/s performed at a fixed temperature of 40° C. Total duration of this step ˜8 min.

The total duration of the full procedure including the sample loading is about 30 min.

The data is analyzed fitting the data to a Three component model [3 component model article citation below] combined with a power law model described by the following expression:

$$σ = {σ}_{y} + {σ}_{y} \cdot {( \frac{\dot{γ}}{{\dot{γ}}_{c}} )}^{0.5} + {K}_{PL} \cdot {\dot{γ}}^{{n}_{PL}}$$

In the expression:
σ: is the shear stress required to maintain a target shear rate \dot{γ}
σ_{y}: is the yield stress
\dot{γ}_{c}: is the critical shear rate
K_{PL}: the consistency parameter describing the viscosity of the continuous phase
n_{PL}: is the power law index describing the shear thinning behavior of the continuous phase

The data obtained from test 1, room temperature data, and test 3, 40C data, are fitted vis standard non linear regression algorithm to estimate the best set of model parameters that fitted the data.

Caggioni, Marco, Veronique Trappe, and Patrick T. Spicer. “Variations of the Herschel-Bulkley exponent reflecting contributions of the viscous continuous phase to the shear rate-dependent stress of soft glassy materials.” Journal of Rheology 64.2 (2020): 413422. incorporated herein by reference.

### Examples

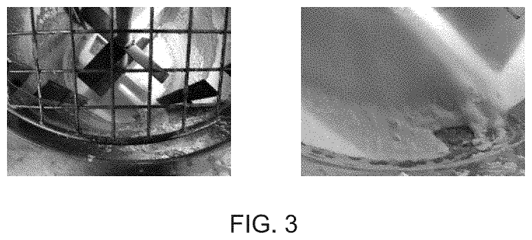

1) Building yield stress (via addition of particulate stabilizing agents) before sulfur addition.
   If sufficient yield stress is not built before sulfur introduction, the sulfur tends to settle out of the batch while being agitated. This manifests as low sulfur yield in finished product and large amounts of unincorporated sulfur residue in making tanks.
   FIG. 3 is two photographs demonstrating examples of unincorporated sulfur left in a tank.
   In the present invention, a composition may be provided, before sulfur addition, which comprises a surfactant, a stabilizing agent(s) and water, wherein the composition comprises at least about 3.0% surfactant and a stabilizing agent(s) sufficient to build a yield stress of at least 0.06 Pa

TABLE 1
Sulfur Yield vs. Order of Addition
This table demonstrates the expected sulfur
yield against different orders of addition.

| | |
|---|---|
| Sulfur yield (fraction) | Sulfur yield (fraction) |
| by HPLC (shampoos | by HPLC (shampoos |
| made by addition of | made by addition of |
| sulfur before addition | sulfur after addition |
| of stabilizers) | of stabilizers) |

| Target | Actual | Target | Actual |
|---|---|---|---|
| 0.95-1.05 | 0.936 | 0.95-1.05 | 1.02 |
| | 0.86 | | 1.025 |
| | 0.928 | | 1.015 |
| | 0.92 | | |
| | 0.945 | | |
| | 0.934 | | |

2) Agitator tip speed at and after the point of sulfur addition (after building yield stress) is above about 1.5 m/s and below about 2.7 m/s with tank recirculation
   a. Too great a mix speed and large quantities of air becomes entrapped in the formula causing low as-made specific gravity (SG<0.98) in finished product followed by unrecoverable phase separation in finished product regardless of final yield—as demonstrated in Example 5 below.
   b. Too low a speed fails to suspend sulfur even with enough yield stress in the system and can result in low sulfur yield and/or finished product phase separation—as demonstrated in Example 4 below.
      Examples of processing using conventional methods:
1.) Common processing techniques of personal care compositions
   a. Uncontrolled or under controlled mix rates—results in inhomogeneity and/or low sulfur yield
   b. Uncontrolled or under controlled recirculation and recirculation rate—results in low sulfur yield
2.) Common processing techniques of particulate containing personal care compositions
   a. Order of addition of stabilizing agents relative to particulate not considered—results in inhomogeneity by overmixing
   b. Method of particulate addition by premix where the premix does not contain stabilizers—results in inhomogeneity of premix and often accompanied by low finished product particulate yield RESULTS

TABLE 2
The below examples demonstrate data generated as sulfur
is added in the process of making a personal care composition
wherein the sulfur is added either before or after stability
agents and various agitator tip speeds.

| | Sulfur Order of | Agitator Tip | % Yield on | |
|---|---|---|---|---|
| Example | Addition (OOA) | Speed (m/s) | Sulfur | Stability |
| 1 | After stability agents | 2.52 | 0.99 | Pass |
| 2 | After stability agents | 1.60 | 0.99 | pass |
| 3 | before stability agents | 2.50 | 0.90 | Fail |
| 4 | After stability agents | 0.78 | 0.84 | Fail |
| 5 | After stability agents | 4.61 | 1.00 | Fail |

This data in Table 2 demonstrates that at the proper order sulfur order of addition and agitator tip speed, a personal care composition will demonstrate proper yield and stability.

### Preparation of Personal Care Compositions

The personal care compositions be prepared from premix or directly as in-line processing. When prepared by premix, the composition is prepared by adding surfactants, anti-dandruff agents, stability agents (as defined previously) and the remainder as water in a particular order and with agitation and recirculation as defined in claim 1. The premix is then combined with perfume, viscosity modifiers, cationic polymers and the remainder of the water with ample agitation to ensure a homogenous and shelf stable mixture.

When done as in-line process, the personal care compositions are prepared by adding surfactants, anti-dandruff agents, stability agents (as defined previously), perfume, viscosity modifiers, cationic polymers and the remainder of the water in an order and with agitation and recirculation as defined in claim 1 to ensure a homogenous and shelf stable mixture. In either case, The mixture can be heated to 50-75° C. to speed the solubilization of the soluble agents, then cooled. Product pH may be adjusted as necessary to provide shampoo compositions of the present invention which are suitable for application to human hair and scalp, and may vary from about pH 4 to 9, or from about pH 4.5 to 6.5, or from about pH 5 to 6, based on the selection of particular detersive surfactants and/or other components.

### Combinations

A. A method for preparing a personal care composition comprising sulfur, wherein the method comprises:
   a) Providing a composition comprising a surfactant, a stabilizing agent(s) and water, wherein the composition comprises at least about 3.0% surfactant and a stabilizing agent(s) sufficient to build a yield stress of at least 0.06 Pa;
   b) Mixing the composition in Step (a) wherein an agitation mechanism tip speed (as calculated by {agitator outer circumference [m/revolution]}*{agitator revolution rate [revolutions/s]}) is between about 1.5 m/s and about 2.7 m/s; with a recirculation turnover rate (as calculated by {recirculation pump rate [L/hour]}/{full turnover volume [L/turnover]}) of greater than 1.8 turnovers/hour;
   c) Adding sulfur to the composition of Step (b)—wherein the conditions of Step b are maintained while adding sulfur and until the personal care composition is prepared.
B. A method according to Paragraph A, wherein the stabilizing agent is selected from the group consisting of homopolymers based on acrylic acid, methacrylic acid or other related derivatives, polyacrylate, polymethacrylate, polyethylacrylate, and polyacrylamide and mixtures thereof.
C. A method according to Paragraph A-B, wherein the stabilizing agent is selected from the group consisting of alkali swellable and hydrophobically-modified alkali swellable acrylic copolymers or methacrylate copolymers, non-limiting examples include acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer.
D. A method according to Paragraph A-C, wherein the stabilizing agent is selected from group consisting of acrylamide/ammonium acrylate copolymer (and) polyisobutene (and) polysorbate 20; acrylamide/sodium acryloyldimethyl taurate copolymer/isohexadecane/polysorbate 80, ammonium acryloyldimethyltaurate/VP copolymer, Sodium Acrylate/Sodium Acryloyldimethyl Taurate Copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; carbomer, sodium carbomer, crosslinked polyvinylpyrrolidone (PVP), polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and) trideceth-6, Acrylamide/Sodium Acryloyldimethyltaurate/Acrylic Acid Copolymer, sodium acrylate/acryloyldimethyltaurate/dimethylacrylamide, crosspolymer (and) isohexadecane (and) polysorbate 60, sodium polyacrylate.
E. A method according to Paragraph A-D, wherein the stabilizing agent is selected from the group consisting of monoesters and/or diesters of ethylene glycol, propylene glycol, diethylene glycol, dipropylene glycol, triethylene glycol or tetraethylene glycol with fatty acids containing from about 6 to about 22, from about 12 to about 18 carbon atoms, such as caproic acid, caprylic acid, 2-ethyhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, arachic acid, gadoleic acid, behenic acid, erucic acid, and mixtures thereof.
F. A method according to Paragraph A-E, wherein the stabilizing agent is selected from the group consisting of ethylene glycol monostearate (EGMS) and/or ethylene glycol distearate (EGDS) and/or polyethylene glycol monostearate (PGMS) and/or polyethyleneglycol distearate (PGDS).
G. A method according to Paragraph A-F, wherein the stabilizing agent is trihydroxystearin.
H. A method according to Paragraph A-G, wherein the composition in 1a) comprises from about 3% to about 20% by weight of a surfactant.
I. A method according to Paragraph A-H, wherein the composition in 1a) comprises from about 3% to about 9% by weight of a surfactant.
J. A method according to Paragraph A-I, wherein the premixture has a temperature of not greater than about 60° C.

It will be appreciated that other modifications of the present disclosure are within the skill of those in the personal care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified. A level of perfume and/or preservatives may also be included in the following examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for preparing a personal care composition comprising sulfur, wherein the method comprises:
a) Providing a composition comprising a surfactant, a stabilizing agent(s) and water, wherein the composition comprises at least about 3.0% surfactant and a stabilizing agent(s) sufficient to build a yield stress of at least 0.06 Pa;
b) Mixing the composition in Step (a) wherein an agitation mechanism tip speed (as calculated by {agitator outer circumference [m/revolution]}*{agitator revolution rate [revolutions/s]}) is between about 1.5 m/s and about 2.7 m/s; with a recirculation turnover rate (as calculated by {recirculation pump rate [L/hour]}/{full turnover volume [L/turnover]}) of greater than 1.8 turnovers/hour;
c) Adding sulfur to the composition of Step (b)—wherein the conditions of Step (b) are maintained while adding sulfur and until the personal care composition is prepared.

2. A method according to claim 1 wherein the stabilizing agent is selected from the group consisting of homopolymers based on acrylic acid, methacrylic acid or other related derivatives, polyacrylate, polymethacrylate, polyethylacrylate, and polyacrylamide and mixtures thereof.

3. A method according to claim 2 wherein the stabilizing agent is selected from the group consisting of alkali swellable and hydrophobically-modified alkali swellable acrylic copolymers or methacrylate copolymers, non-limiting examples include acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer.

4. A method according to claim 3 wherein the stabilizing agent is selected from group consisting of acrylamide/ammonium acrylate copolymer (and) polyisobutene (and) polysorbate 20; acrylamide/sodium acryloyldimethyl taurate copolymer/isohexadecane/polysorbate 80, ammonium acryloyldimethyltaurate/VP copolymer, Sodium Acrylate/Sodium Acryloyldimethyl Taurate Copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; carbomer, sodium carbomer, crosslinked polyvinylpyrrolidone (PVP), polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and) trideceth-6, Acrylamide/Sodium Acryloyldimethyltaurate/Acrylic Acid Copolymer, sodium acrylate/acryloyldimethyltaurate/dimethylacrylamide, crosspolymer (and) isohexadecane (and) polysorbate 60, sodium polyacrylate.

5. A method according to claim 1 wherein the stabilizing agent is selected from the group consisting of monoesters and/or diesters of ethylene glycol, propylene glycol, diethylene glycol, dipropylene glycol, triethylene glycol or tetraethylene glycol with fatty acids containing from about 6 to about 22, from about 12 to about 18 carbon atoms, such as caproic acid, caprylic acid, 2-ethyhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, arachic acid, gadoleic acid, behenic acid, erucic acid, and mixtures thereof.

6. A method according to claim 1 wherein the stabilizing agent is selected from the group consisting of ethylene glycol monostearate (EGMS) and/or ethylene glycol distearate (EGDS) and/or polyethylene glycol monostearate (PGMS) and/or polyethyleneglycol distearate (PGDS).

7. A method according to claim 1 wherein the stabilizing agent is trihydroxystearin.

8. A method according to claim 1 wherein the composition in 1a) comprises from about 3% to about 20% by weight of a surfactant.

9. A method according to claim 1 wherein the composition in 1a) comprises from about 3% to about 9% by weight of a surfactant.

10. A composition according to claim 1 wherein the premixture has a temperature of not greater than about 60° C.
